(19) United States
(12) Patent Application Publication
(10) Pub. No.: US 2015/0366622 A1
(43) Pub. Date: Dec. 24, 2015

(54) DEVICE FOR THE SELF-LOCKING BIDIRECTIONAL DRIVE OF A MEDICAL TREATMENT DEVICE

(71) Applicant: Maquet GmbH, Rastatt (DE)

(72) Inventors: Ulrich Wyslucha, Weingarten (DE); Dominik Biel, Kuppenheim (DE); Mike Obert, Gernsbach (DE)

(21) Appl. No.: 14/745,946

(22) Filed: Jun. 22, 2015

(30) Foreign Application Priority Data
Jun. 23, 2014 (DE) 10 2014 108 745.3

(51) Int. Cl. A61B 19/00; A61G 13/12

(52) U.S. Cl. CPC A61B 19/203; A61G 13/1245

(57) ABSTRACT

The invention relates to a device for the self-locking bidirectional drive of a medical treatment device (18) with a first locking unit for blocking the rotation of an inner part (72) in a first direction of rotation (R1) and for releasing the rotation of the inner part (72) in a second direction of rotation (R2) and comprising a second locking unit for blocking the rotation of the inner part (72) in a second direction of rotation (R2) and for releasing the rotation of the inner part (72) in a first direction of rotation (R1). The inner part (72) is connected to the medical treatment device (18) via a driven shaft (55). Two release elements (122a to 122c, 123a to 123c) are provided which are movable by means of a drive element (36) into one release position each, which release elements prevent the blocking of the rotation of the inner part (72) in one direction of rotation (R1, R2) each so that a rotation of the inner part (72) and of the medical treatment device (18) connected to the inner part (72) via the driven shaft (55) is only possible by means of the drive element (36).

### CROSS REFERENCE TO RELATED APPLICATIONS

Applicant hereby claims foreign priority benefits under U.S.C. §119 from German Application No. DE 10 2014 108 745.3 filed on Jun. 23, 2014, the contents of which are incorporated by reference herein.

### TECHNICAL FIELD

The invention relates to a device for the self-locking bidirectional drive of a medical treatment device, comprising an inner part rotatable about an axis of rotation, a rotationally-fixed outer part having a circular opening which surrounds the inner part and is arranged concentrically about the axis of rotation, a driven shaft rotatable about the axis of rotation and connectable to the inner part and to the treatment device, and comprising a drive element for rotating the inner part together with the driven shaft. Further, the device comprises a first clamping part and a second clamping part which are arranged in an intermediate space between the inner part and the outer part.

### BACKGROUND

During the treatment of patients it is often necessary that parts of the patient, in particular limbs have to be fixed and moved in a well-directed manner. In particular, during operations in which the position of a body part has to be changed in a well-directed manner several times, adjustable treatment devices are necessary. Thus, in the case of a hip prosthesis procedure according to the direct interior approach as well as in the case of the total hip arthroplasty a patient's leg has to be rotated repeatedly. Here, the straight patient's leg is fixed in a self-supporting manner in a traction unit. In connection with operating tables, such traction units are also referred to as extension devices. The angle of rotation of the leg then has to be changed several times in accordance with the surgical work flow and the medical needs. The maximum normal rotational range amounts to 180°.

For changing the angle of rotation of a patient's leg fixed in an extension device, an operator has to loosen a clamping screw for releasing the rotation so that the rotation of the patient's leg is released by the extension device. Thereafter, the operating surgeon rotates the patient's leg into the desired position. Here, the tip of the foot of the patient can be used as an indicator for the angle of rotation. The patient's leg then has to be held manually in the desired angle of rotation, wherein by way of the anatomical structure of the leg a restoring force into an angle of rotation deviating from the desired angle of rotation occurs. Subsequently, the clamping screw is tightened so that the patient's leg is fixed by the extension device in the desired angle of rotation. To bring the patient's leg back into the previous position or to bring it into another angle of rotation, the clamping screw has to be loosened again, the patient's leg has to be rotated back again in a controlled manner or has to be rotated into the desired position. Thereafter, the clamping screw is tightened again.

Dependent on the surgical workflow, this procedure has to be repeated several times during an operation. In the described course, the actions of loosening/tightening of the clamping screw and rotating/holding of the patient's leg in the desired position have to be performed simultaneously to some extent. In this connection, the restoring effort of the patient's leg into a defined position is often aggravating and results in a considerable stress for the operating surgeon since considerable force and concentration are necessary for the coordination of the required working steps. A sensitive positioning of the patient's leg during an operation is thus only possible in a restricted manner so that as a result thereof this represents a risk for the patient during the course of the operation. To keep this risk in limits, it is common practice that one person loosens and tightens the clamping screw and a second person brings the patient's leg into the desired position and holds it thereat until the clamping screw has been tightened again. With two people, the expenditure for this is relatively high.

### SUMMARY

It is the object of the invention to specify a device with which the positon of a medical treatment device can be changed easily and safely.

This object is solved by a device for the self-locking bidirectional drive of a medical treatment device comprising an inner part rotatable about an axis of rotation (Z), a rotationally-fixed outer part having a circular opening which surrounds the inner part and is arranged concentrically about the axis of rotation (Z), a driven shaft rotatable about the axis of rotation (Z), which driven shaft is connected to the inner part and is connectable to the treatment device, a drive element for rotating the inner part together with the driven shaft about the axis of rotation (Z), with a first clamping part and with a second clamping part which are arranged in an intermediate space between the inner part and the outer part, wherein the outer part, the inner part and the first clamping part form a first locking unit for blocking the rotation of the inner part in a first direction of rotation (R1) and for releasing the rotation of the inner part in a second direction of rotation (R2), that the outer part, the inner part and the second clamping part form a second locking unit for blocking the rotation of the inner part in the second direction of rotation (R2) and for releasing the rotation of the inner part in the first direction of rotation (R1), that a first release element movable into a first release position by the drive element is provided, which release element prevents the blocking of the rotation of the inner part in the first direction of rotation (R1) by the first locking unit, and that a second release element movable into a second release position by the drive element is provided, which release element prevents the blocking of the rotation of the inner part in the second direction of rotation (R2) by the second locking unit.

By way of the locking units for locking the rotation of the inner part, which locking units act in opposite rotational directions, a rotation of the driven shaft and thus also a rotation of a treatment device connected to the driven shaft, such as a foot receptacle for receiving the foot of a patient's leg, is prevented when a driven-side or output-side torque is applied. By way of the inventive device for the self-locking bidirectional drive of a medical treatment device a rotation of the inner part and thus of the driven shaft is only possible by means of the drive element since this drive element moves the first release element for releasing the first direction of rotation as well as the second release element for releasing the second direction of rotation. As a result, a rotation of the driven shaft and thus an adjustment of the medical treatment device is easily possible by actuating the drive element, which makes an easy handling of the medical treatment device, in particular an actuation by one person only, easily possible. In particular, clamping or locking elements which are to be actuated separately can be dispensed with, since a locking is automatically guaranteed by means of the locking units acting in opposite directions of rotation. Further, by means of the drive element an exact rotation of the driven shaft is easily possible so that desired angular positions of the driven shaft and of the treatment device connected to the driven shaft can be set in an easy manner.

It is particularly advantageous when a rotating spindle drive is additionally provided, by which the medial treatment device is movable in the direction of the axis of rotation or parallel to the axis of rotation. As a result thereof, the medical treatment device can be moved along the axis of rotation or parallel to the axis of rotation in longitudinal direction and, additionally, the angle of rotation of the treatment device can be set precisely by means of the drive element in order to exert, for example, a tractive force on the patient's leg. The rotating spindle of the rotating spindle drive is preferably passed centrally through the driven shaft, the rotating spindle being arranged freely rotatably with respect to the driven shaft.

It is particularly advantageous when the drive element, when rotated in the first direction of rotation, at first moves the first release element from a first neutral position into the first release position in which the first release element prevents the blocking of the rotation of the inner part in the first direction of rotation by the first locking unit and, when rotated further, rotates the inner part in the first direction of rotation. Further, the drive element, when rotated in the second direction of rotation, at first moves the second release element from a second neutral position into the second release position in which the second release element prevents the blocking of the rotation of the inner part in the second direction of rotation by the second locking unit and, when rotated further in the second direction of rotation, rotates the inner part in the second direction of rotation. Thus, when the drive element is rotated in the first direction of rotation, at first a release of the rotation of the inner part in the first direction of rotation or the unblocking of the blocking of the rotation of the inner part in the first direction of rotation and thereafter the rotation of the inner part and of the driven shaft coupled to the inner part takes place. Likewise, when the drive element is rotated in the second direction of rotation, at first a release of the rotation of the inner part in the second direction of rotation and, when the drive element is rotated further in the second direction of rotation, a rotation of the inner part in the second direction of rotation takes place. Preferably, the first release element and the second release element are automatically moved back from the first release position and the second release positon, respectively, into the first and the second neutral position, respectively, preferably they are moved by means of a spring force from the respective release position into the neutral position. As a result, an easy handling of the device without changing the desired direction of rotation or unlocking is possible.

Further, it is advantageous when the first clamping part is clampable in a first clamping area formed between the outer part and the inner part when the inner part is rotated in the first direction of rotation, and when the second clamping part is clampable in a second clamping area formed between the outer part and the inner part when the inner part is rotated in the second direction of rotation. The clamping of the first clamping part in the first clamping area and of the second clamping part in the second clamping area, respectively, only takes place when the first release element and the second release element, respectively, are not in their release position. By clamping the clamping parts in the second clamping area, it is guaranteed that a blocking of the rotary motion in the first direction of rotation and in the second direction of rotation only takes place when the release elements have not been moved into their release position by means of the drive unit. Thus, a rotation of the driven shaft via the medical treatment device is prevented easily and effectively without specific actions, such as the tightening of clamping screws or the like, being necessary for this. Such clamping arrangements with clamping parts which are arranged between an outer part and an inner part and which are clamped in a clamping area formed between the outer part and the inner part are, for example, known from so-called sprag clutches which enable a free rotation of an inner part relative to an outer part in a first direction of rotation and which establish a connection between the inner part and the outer part when rotated in the other direction of rotation.

By clamping the clamping parts in the corresponding clamping area, a simple and almost maintenance-free arrangement is provided which reliably prevents an undesired rotation of the medical treatment device about the axis of rotation.

Further, it is advantageous when an elastically deformable element is arranged between the first clamping part and the second clamping part, which element presses the first clamping part into the first clamping area and the second clamping part into the second clamping area. As a result, the clamping parts are safely arranged in the respective clamping area so that a blocking of the rotary motion free from play is possible and, as a result, a stable exact positioning of the medical treatment device is achieved.

It is particularly advantageous when the elastic element is a spring, preferably a coil spring arranged between the clamping parts. Alternatively, the elastically deformable element can be formed by an elastomer block or polymer block. By means of these elements it can easily be guaranteed that the clamping parts are pressed into their respective clamping area and are safely held thereat. Further, the respective clamping part can be pushed out of the clamping area against the spring force when the respective release element is moved into the release position so that the movement is no longer blocked by the respective clamping part.

Further, it is advantageous when the first release element is arranged on the side of the first clamping part facing away from the elastically deformable element and the second release element is arranged on the side of the second clamping part facing away from the elastically deformable element. As a result, an easy and compact arrangement of the clamping parts and the release elements is possible.

Further, it is advantageous when the first locking unit and the second locking unit prevent a rotation of the inner part and of the driven shaft without an actuation of the drive element when an output-side torque is applied to the driven shaft and/or to the inner part. As a result, an easy and safe handling of the treatment device is made possible since also in the case of an output-side torque applied to the inner part a rotation of the inner part and of the driven shaft is reliably prevented without an actuation of the drive element. Thus, an easy and safe handling of the treatment device is possible.

Further, it is advantageous when the device enables a rotation of the driven shaft only by the actuation of the drive element. Thus, it is guaranteed that a rotation of the medical treatment device by means of an output-side torque is safely prevented and thus a safe handling of the medical treatment device is possible.

Further, it is advantageous when the drive element is engaged with the inner part via at least one engagement element, wherein the engagement element is received with play in a recess of the inner part and/or wherein the engagement element is received with play in a recess of the drive element. The play preferably has a value in the range between 0.5 mm and 5 mm or between 0.01° to 2°.

Specifically, the engagement element can be designed as a pin and the recess can be designed as a bore, wherein the pin projects into the bore and the diameter of the bore is preferably larger than the diameter of the pin by a value in the range between 0.5 mm to 5 mm.

The drive element is preferably directly, i.e. free from play, coupled to the release elements or is directly engaged with these free from play. As a result, it can easily be guaranteed that at first the first release element is moved into the first release position so that the locking of the first locking unit is prevented and that thereafter a rotation of the inner part in a first direction of rotation takes place. Likewise, when the drive unit is rotated in the second direction of rotation, at first the second release element is moved into the second release position in which the second release element prevents a clamping of the second clamping part in the second clamping area, and thereafter a rotation of the inner part by the drive unit in the second direction of rotation takes place. Thus, neither a change in the direction of rotation nor an additional locking is required. Merely, a drive by means of the drive element has to take place to release and perform the desired rotary motion in one operating action.

It is particularly advantageous when the first release element and the second release element are operatively connected with an actuating element, and when the actuating element, when actuated, moves the first release element into the first release position and the second release element into the second release position. As a result, the locking by means of the first locking unit and the locking by means of the second locking unit can be unlocked simultaneously independent of the actuation of the drive element so that then a rotation of the driven shaft and of the medical treatment device connected to the driven shaft can also take place on the output side. Thus, a release of the rotation takes place by the actuation of the actuating element. By releasing the rotation, the medical treatment device can be freely rotated, for example to judge the function and the movability of joints of a limb of a patient connected to the treatment device, such as the rotation of a patient's leg after the insertion of a hip endoprosthesis or an artificial knee joint.

Further, it is advantageous when a first locking and release arrangement comprises at least the first locking unit, the second locking unit, the first release unit and the second release unit and when the device comprises at least a second locking and release arrangement, wherein the structure and the function of the second locking and release arrangement corresponds to the structure of the first locking and release arrangement. Thus, the second locking and release arrangement can comprise at least a third locking unit, a fourth locking unit, a third release unit and a fourth release unit. The at least two locking and release arrangements are arranged about the axis of rotation at equal angular distances. Thus, the forces occurring between the outer part and the inner part for blocking the movement are uniformly distributed over the circumference of the inner part and the outer part so that the forces acting on the inner part by the locking and release arrangements orthogonally to the axis of rotation are preferably zero when added together. As a result, neither the inner part nor the driven shaft have to take up lateral forces by the locking and release arrangements, as a result whereof a simple and compact design is possible. It is particularly advantageous when the device comprises three locking and release arrangements, the structure and function of which correspond to each other and which are arranged about the axis of rotation at equal angular distances. As a result, a particularly simple and compact structure is possible.

It is particularly advantageous when the first locking and release arrangement comprises at least the first release element, the second release element, the first clamping part and the second clamping part, when the second locking and release arrangement comprises at least a third release element, a fourth release element, a third clamping part and a fourth clamping part, when the third locking and release arrangement comprises at least a fifth release element, a sixth release element, a fifth clamping part and a sixth clamping part, when the first, third and fifth release element are connected to each other such that they are jointly movable about the axis of rotation and when the second, fourth and sixth release element are connected to each other such that they are jointly movable about the axis of rotation. The first, third, and fifth release element are jointly movable relative to the second, fourth and sixth release element, preferably about an angle in the range between 0.5° and 5°. As a result, a common movement of the first, third and fifth release element relative to the second, fourth and sixth release element is possible so that such a movement can be generated by means of the actuating element, as a result whereof all release elements can simultaneously be moved into their respective release position and a clamping of the respective clamping part in the respective clamping area is easily prevented. In this way, the complete unlocking of the locking effect of the first locking unit and of the second locking unit or of corresponding locking units of the further locking and release arrangements can be achieved easily by actuating the actuating element.

Further, it is advantageous when the actuating element, when actuated, moves the first, third and fifth release element jointly in the first direction of rotation and the second, fourth and sixth release element in the second direction of rotation such that the first release element contacts the first clamping part and prevents a clamping of the first clamping part in the first clamping area, that the second release element contacts the second clamping part and prevents a clamping of the second clamping part in the second clamping area, that the third release element contacts a third clamping part of the second locking and release arrangement and prevents a clamping of the third clamping part in a third clamping area formed between the inner part and the outer part, that the fourth release element contacts a fourth clamping part of the second locking and release arrangement and prevents a clamping of the fourth clamping part in a fourth clamping area formed between the inner part and the outer part, that the fifth release element contacts a fifth clamping part of the third locking and release arrangement and prevents a clamping of the fifth clamping part in a fifth clamping area formed between the inner part and the outer part, and/or that the sixth release element contacts a sixth clamping part of the third locking and release arrangement and prevents a clamping of the sixth clamping part in a clamping area formed between the inner part and the outer part. As a result, an easy complete release of the rotation takes place so that then a rotation is also possible by means of an output-side torque. The locking effect of the respective locking units is thus released by an actuation of the actuating element. The actuating element can be designed such that it remains in the actuated state until it is moved from the actuated state into the non-actuated state by an operator. Alternatively, the actuating element can also be moved from the actuated state back into the non-actuated state by a restoring force. In this case, an operator would have to hold the actuating element in the actuated state as long as the rotation of the driven shaft shall be released. The restoring can be effected by means of a spring.

Preferably, the clamping parts are designed as clamping rolls, the longitudinal axes of which are arranged parallel to the axis of rotation. The cross-sectional area of the inner part in a plane transversely to the axis of rotation is substantially the one of an equal-sided triangle.

The drive element is preferably designed as a handwheel which is rotated about the axis of rotation. Preferably, the handwheel is directly connected to the engagement elements for actuating the release elements and for rotating the inner part. Alternatively, a gear stage, preferably with a gear reduction, can be provided between the handwheel and the engagement elements.

As a result, the operating surgeon can, by means of the invention, rotate in particular a patient's leg connected to the medical treatment device by means of the drive unit in the desired direction and set a desired angular position, wherein at the same time a restoring movement of the patient's leg is automatically prevented by the locking units. The locking units act oppositely to each other. By enabling the unlocked rotation of the medical treatment device by actuating the actuating element, in particular a function control in the case of hip joint operations, in particular a function control of an inserted hip endoprosthesis is made possible. A switching between the locked rotation by means of the locking units and the release by means of the actuating element is possible intraoperatively by one operator only, in particular the operating surgeon, without any difficulties by actuating the actuating element.

By means of the automatic blocking of a restoring movement, a considerable relief for the operator, in particular the operating surgeon, is given. Further, in this way, the precision of the positioning of the medical treatment device in a desired angular position can be increased. Also the correction of the angle of rotation by small angular amounts is easily possible simply by the continuous adjustment and automatic blocking of the rotary motion. Also the risk of unintended changes of position of the medical treatment device is prevented by the automatic blocking of the rotary motion by means of the locking units. The surgical workflow during an operation or treatment of a patient is simplified and accelerated as less actions are required as compared to known clamping devices for setting the rotations of the medical treatment device.

### BRIEF DESCRITION OF THE DRAWINGS

Further features and advantages of the invention result from the following description which explains the invention in more detail on the basis of embodiments in connection with the enclosed Figures.

FIG. 1 shows a perspective illustration of an operating table with an extension set for hip arthroscopy and minimal invasive hip endoprosthesis, which comprises an adjusting unit for rotation and length adjustment of a foot receptacle.

FIG. 2 shows a perspective side view of the adjusting unit.

FIG. 3 shows a longitudinal section of the adjusting unit.

FIG. 4 shows an enlarged illustration of a detail of the longitudinal section according to FIG. 3 with elements of a rotary drive.

FIG. 5a shows a side view of an arrangement of selected elements of locking and release units of the rotary drive according to FIG. 4.

FIG. 5b shows a sectional view of the arrangement according to FIG. 5a along the sectional line A-A, wherein an outer part of the rotary drive is additionally illustrated.

FIG. 5c shows a sectional view of the arrangement according to FIG. 5a along the sectional line B-B.

FIG. 6 shows a perspective illustration of a cross-section of the rotary drive along the sectional line A-A indicated in FIG. 5a.

FIG. 7a shows a further view of an arrangement of elements of the rotary drive in a partially sectional illustration.

FIG. 7b shows a cross-section of the arrangement according to FIG. 7a along the sectional line A-A.

FIG. 8 shows a further perspective illustration of elements of the rotary drive in which elements for the release of rotation are well visible, and

FIG. 9 shows a detailed illustration of elements for the release of rotation.

### DETAILED DESCRIPTION

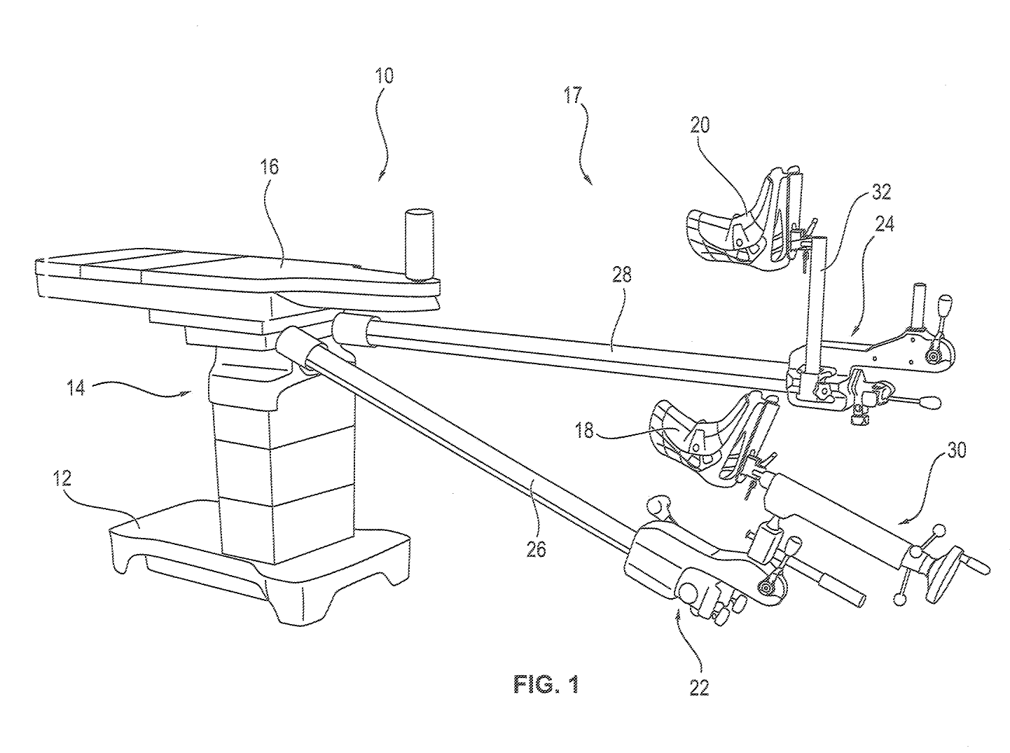

FIG. 1 shows a perspective illustration of an arrangement of an operating table 10 with an extension set 17, which is preferably used for hip arthroscopy and minimal invasive hip endoprosthesis. The operating table 10 has an operating table foot 12, an operating table column 14 and a patient support surface 16.

The extension set 17 comprises a first traction bar 26 and a second traction bar 28 which are each connected to a first end of the operating table 10. At the opposite second end of the first traction bar 26, the traction bar 26 is connected to a first foot receptacle 18 via a first connecting unit 22 and via an adjusting unit 30 for rotation and length adjustment. The second traction bar 28 is connected to a second foot receptacle 20 via a second connecting unit 22.

The first foot receptacle 18 serves to receive the foot of the patient's leg to be operated and the second foot receptacle 20 serves to receive the foot of the patient's leg not to be operated. Thus, an adjusting unit for rotation and length adjustment is not necessary between the second connecting unit 24 and the second foot receptacle 20. The second foot receptacle 20 is connected to a second connecting unit 24 via a connecting rod 32. The connecting units 22, 24 are arranged movably and lockably on the respective traction bars 26, 28 to adapt the extension set 17 to the stature of the patient. The adjusting unit 30 for rotation and length adjustment is also referred to as screw tension device.

Alternatively, an adjusting unit can also be arranged between the connecting unit 24 and the foot receptacle 20, the structure and function of which corresponds to the structure and function of the adjusting unit 30.

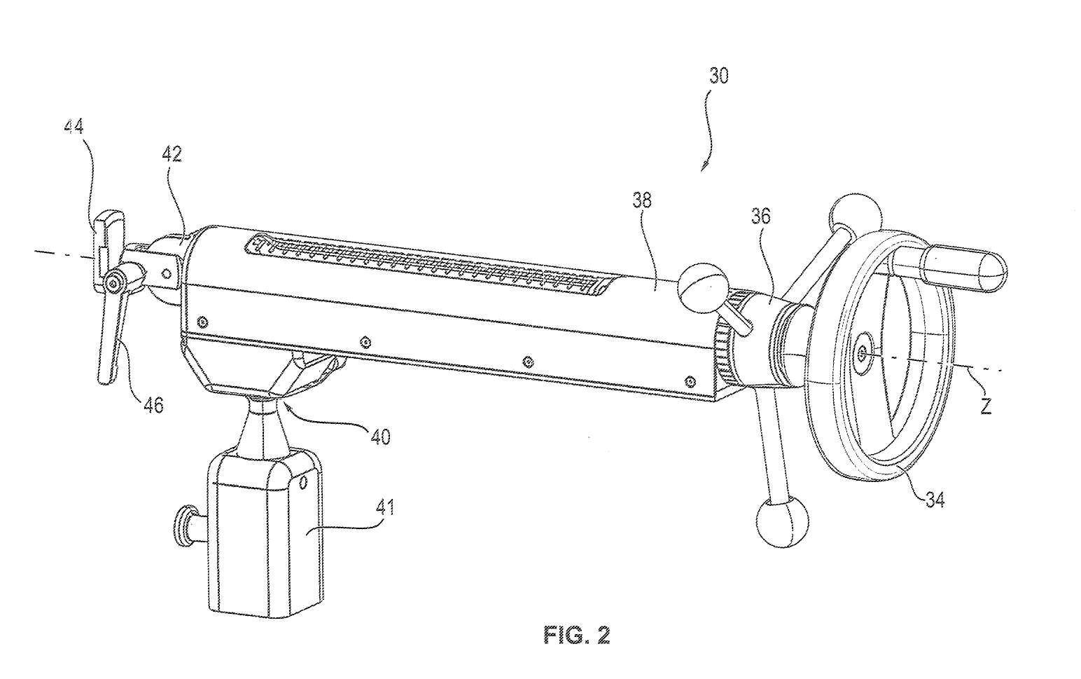

FIG. 2 shows a perspective side view of the adjusting unit 30. The adjusting unit 30 has a first handwheel 34 for the axial movement of a connecting element 44 along a longitudinal and rotational axis Z of the adjusting unit 30. The connecting element 44 serves to connect the adjusting unit 30 to the first foot receptacle 18. The adjusting unit 30 further comprises a second handwheel 36 for the rotation adjustment of the connecting element 44 so that by means of the handwheel 36 the angle of rotation of the first foot receptacle 18 can be changed.

In a housing of the adjusting unit 30, a mechanism operatively connected to the rotary wheels 34, 36 is arranged, with which the connecting element 44 is connected via a positioning head 42. Via the positioning head 42, the position of the connecting element 44 is pivotable about an axis of rotation orthogonally to the longitudinal and rotational axis Z of the adjusting unit 30. By means of a clamping lock 46 of the positioning head 42, the connecting element 44 is lockable in a pivot position. Via an articulation 40, the adjusting unit 30 is connected to an articulated head support 41 of the connecting unit 22.

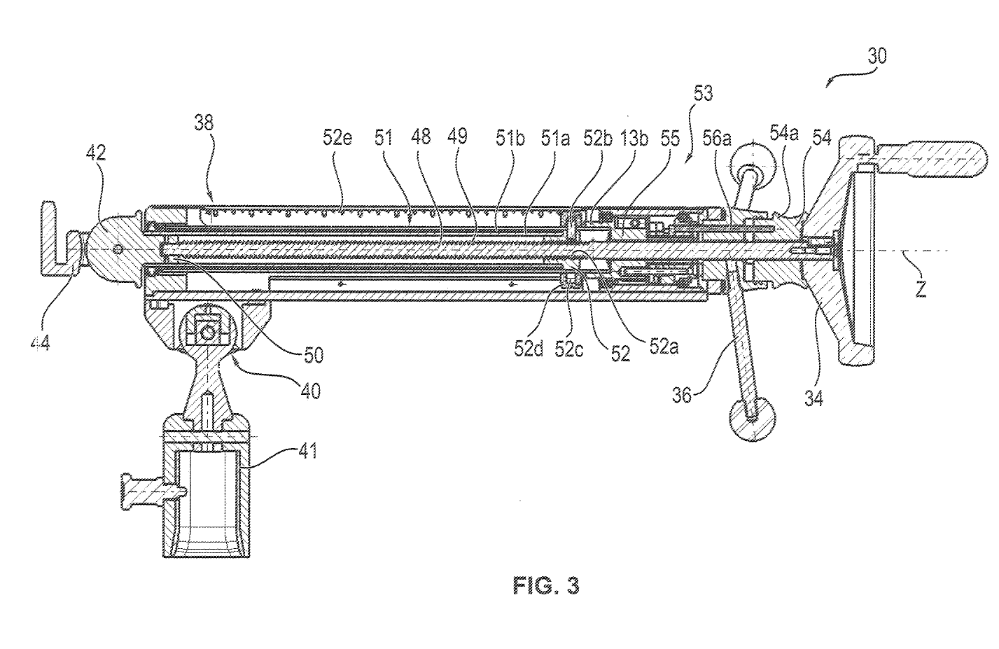

FIG. 3 shows a longitudinal section of an adjusting unit 30. The adjusting unit 30 has a drive spindle 48 which is firmly connected to the first handwheel 34 and serves to drive a telescopic tube arrangement 51 for the axial displacement of the positioning head 42. The telescopic tube arrangement 51 is illustrated in the Figures in a retracted position. The telescopic tube arrangement 51 has an inner telescopic tube 51a and an outer telescopic tube 51b. The outer telescopic tube 51b is received in a rotationally fixed manner in a front-side opening of a driven shaft 55 of a rotary drive 53 driven by means of the second handwheel 36. The structure and the function of the rotary drive 53 will still be explained in more detail in the following in connection with the further Figures.

The end of the inner telescopic tube 51a facing the first drive wheel 34 is firmly connected to a slide 52 which is movable by means of the drive spindle 48 and the internal thread 52a of which is engaged with the external thread 49 of the drive spindle 48. The driving slide 52 has a first nose 52b projecting upward through an upper slot present in the outer telescopic tube 51b and a nose 52c projecting downward through a second lower slot in the outer telescopic tube 51b. As a result, when the outer telescopic tube 51b is rotated by means of the rotary drive 53, the slide 52 is rotated together with the outer telescopic tube 51b so that the inner telescopic tube 51a connected to the slide 52 in a rotationally fixed manner and the positioning head 42 are rotated together with the outer telescopic tube 51b. When extending the inner telescopic tube 51a from the outer telescopic tube 51b, the positioning head 42 is retracted and extended while maintaining its angular position. The end of the drive spindle 48 facing the positioning head 42 is rotatably received in a bearing 50, wherein the drive spindle 48 is connected to the bearing 50 such that the bearing 50 slides along the inner wall of the inner telescopic tube 51a when the latter is retracted and extended. With its end opposite to the driven shaft 55, the outer telescopic tube 51b is received in a bearing bush formed in the housing 38 so that this end of the outer telescopic tube 51b is safely held and rotatable relative to the housing 38. The noses 52b, 52c of the slide 52 are engaged with a circumferential indicator ring 52d via which the axial position of the inner telescopic tube 51a is indicated on a scale 52e visible from outside through an observation window in the housing 38. By the rotation of the drive spindle 48 by means of the first handwheel 34, the inner telescopic tube 51a can be moved out of and again into the outer telescopic tube 51b.

The longitudinal axis Z of the adjusting unit 30 is at the same time the center axis of the drive spindle 48 and of the telescopic tubes 51a, 51b so that the longitudinal axis Z is at the same time the axis of rotation Z about which then the positioning head 42 is rotatable together with the foot receptacle 18 by means of the rotary drive 53.

Between the first handwheel 34 and the second handwheel 36, an actuating element 54 is arranged which, when actuated, enables a rotation of the outer telescopic tube 51b together with the inner telescopic tube 51a also without a rotation of the second handwheel 36. For actuating the actuating element 54, it is displaced along the axis of rotation Z in the direction of the positioning head 42. The actuating stroke along the axis of rotation Z is delimited by the distance of the end of the hub 37 of the second handwheel 36 facing away from the positioning head 42 and a projection 54a provided on the actuating element 54. To enable a rotation independent of the second handwheel 36, the actuating element 54 is engaged via three axially displaceable pins, one pin of which, visible in FIG. 3, is identified with the reference sign 56a. The three pins are arranged at equal angular distances about the axis of rotation Z and are identified in the further Figures with the reference signs 56a, 56b, 56c.

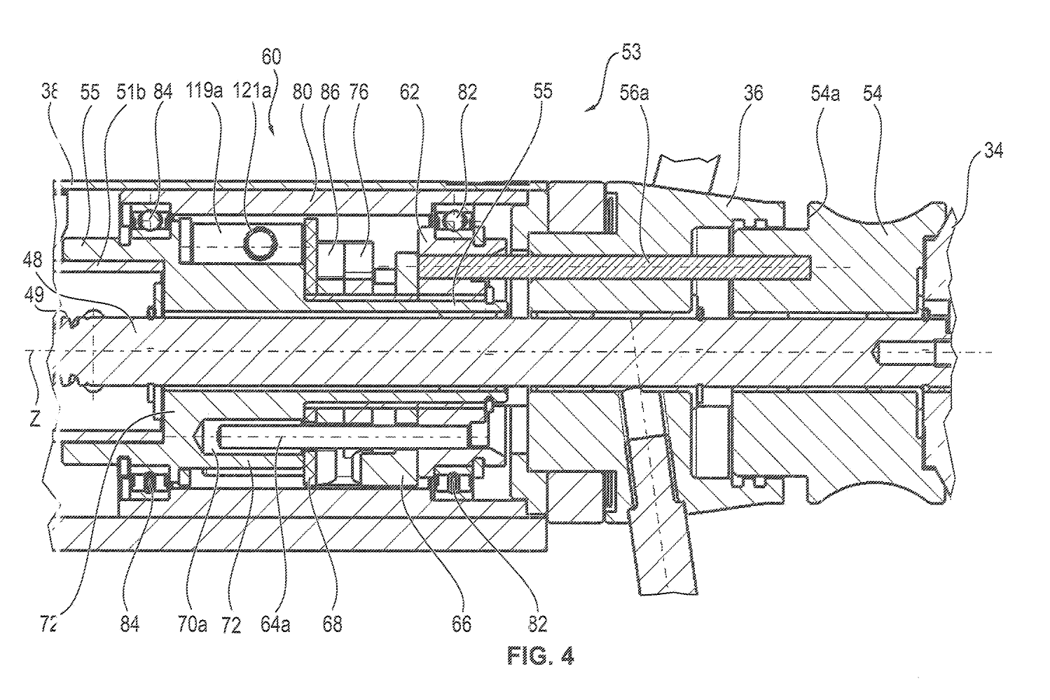

FIG. 4 shows an enlarged illustration of a detail of the longitudinal section according to FIG. 3 with elements of the rotary drive 53. The rotary drive 53 comprises a device 60 for the self-locking bidirectional drive of the driven shaft 55. The second handwheel 36 is engaged with a rotary body 62 via the pins 56a to 56c so that a rotary motion of the second handwheel 36 is transmitted to the rotary body 62. The rotary body 62 is received in the housing 38 via a first ball bearing 82 rotatably about the axis of rotation Z. Further, the rotary body 62 is arranged freely rotatably on the end of the driven shaft 55 facing the second handwheel 36. Further, both the second handwheel 36, the actuating element 54 and the driven shaft 55 are each arranged freely rotatably on the drive spindle 48. Further, the end of the driven shaft 55 facing the positioning head 42 is received in the housing 38 via a second ball bearing 84. Further, three driving pins which are firmly connected to the rotary body 62 and of which one driving pin, visible in FIG. 4, is identified with the reference sign 64a, and the further driving pins, visible in the further Figures, are identified with the reference signs 64b and 64c, project into three bores provided in an area of the driven shaft 55 forming an inner part 72 of the rotary drive 53, of which bores the bore visible in FIG. 4 is identified with the reference sign 70a and the further bores visible in the further Figures are identified with the reference signs 70b and 70c. Here, the driving pins 64a to 64c are passed through a rotation release disc 66, wherein the rotation release disc 66 is movable in the direction of the axis of rotation Z by means of the pins 56a to 56c in the direction of the positioning head 42 for release of the rotation of the driven shaft 55 without actuating the second handwheel 36.

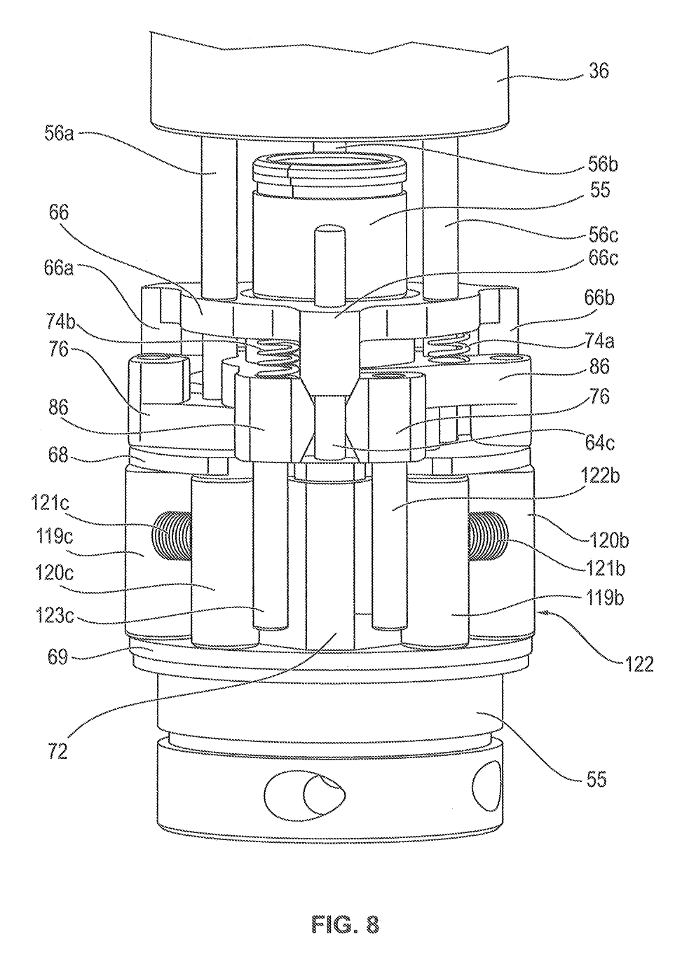

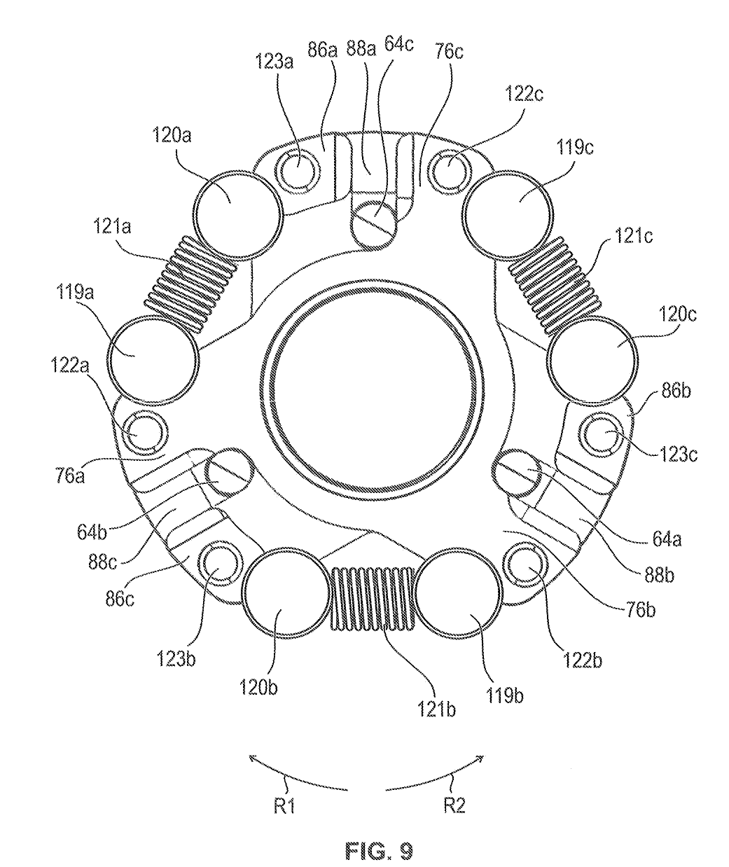

In the present embodiment, the diameter of the bores 70a to 70c is 2 mm greater than the diameter of the driving pins 64a to 64c. Thus, the driving pins 64a to 64c are received in the bores 70a to 70c with a play of 2 mm, i.e. 1 mm in each direction of rotation R1, R2 so that when the second handwheel 36 is rotated no rotation of the driven shaft 55 is caused within this play. As is well visible in FIG. 9, the driving pins 64a to 64c are passed between the arms 76a to 76c of a first rotating spider 76 and between the arms 86a to 86c of a second rotating spider 86, with which release pins serving as release elements are firmly connected. The longitudinal axes of the release pins as well as the longitudinal axes of the driving pins 64a to 64c run parallel to the axis of rotation Z. The release pins connected to the respective rotating spider 76, 86 are rotated by a rotation of the respective rotating spider 76, 86 that is caused by the rotation of the driving pins 64a to 64c within the play present between the bores 70a to 70c and the driving pins 64a to 64c, wherein dependent on the direction of rotation the first rotating spider 76 or the second rotating spider 86 is rotated. When the actuating element 54 is actuated, the rotation release disc 66 is moved in the direction of the inner part 72 via the pins 56a to 56c, as a result whereof both rotating spiders 76, 86 are simultaneously rotated in opposite directions of rotation, as will still be explained in more detail later in connection with FIGS. 8 and 9. Between the inner part 72 and the rotating spiders 76, 86, an intermediate disc 68 provided with openings for the passage of the driving pins 64a to 64c is arranged.

The first rotating spider 76 and the second rotating spider 86 are mounted on the driven shaft 55 rotatably about the axis of rotation Z. Around the inner part 72 an outer part 80 connected to the housing 38 in a rotationally fixed manner is arranged, which outer part has a circular opening 80a at least in the area of the inner part 72. Between the inner part 72 and the outer part 80 altogether six clamping rolls are arranged, of which the clamping roll visible in FIG. 4 is identified with the reference sign 119a.

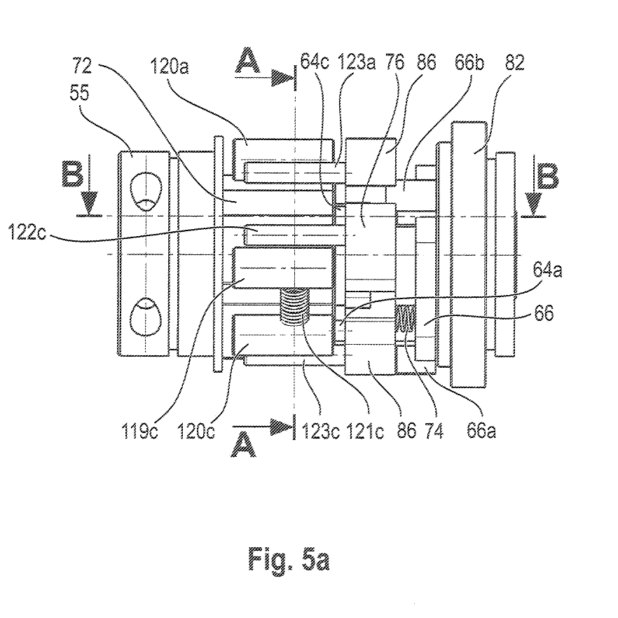

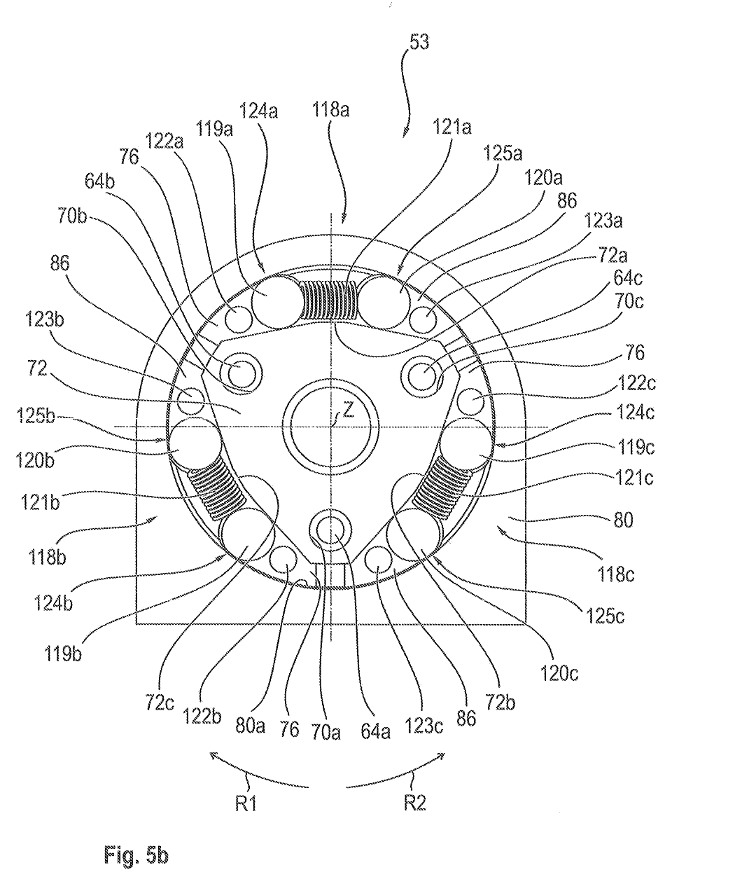

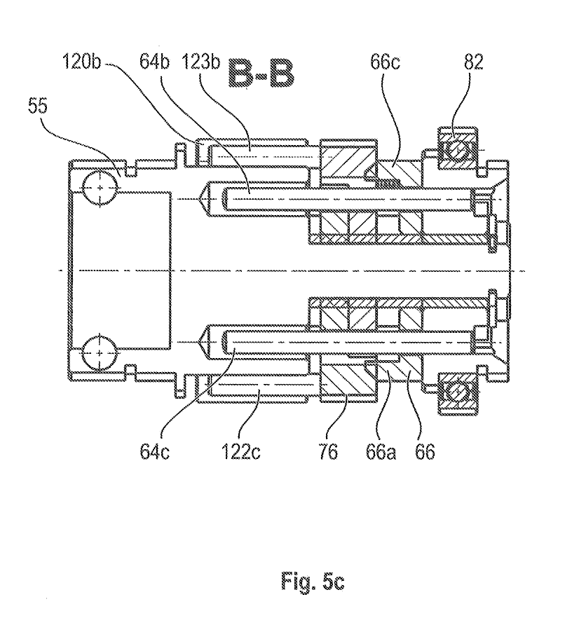

FIG. 5a shows a side view with an arrangement of selected elements of the rotary drive 53, and FIG. 5b shows a cross-section of the arrangement according to FIG. 5a along the sectional line A-A. FIG. 5c shows a sectional view of the arrangement according to FIG. 5a along the sectional line B-B.

The inner part 72 has a substantially triangular basic shape with an equal leg length of the legs 72a to 72c. Further, in FIG. 5b the outer part 80 stationarily arranged in the housing 38 is additionally illustrated. Between the legs 72a to 72c and the inner circular openings 80a of the outer part 80 locking and release arrangements 118a to 118c are arranged about the axis of rotation Z at equal angular distances each time, as can be best seen in FIG. 5b. The elements of the individual locking and release arrangements 118a to 118c are each identified with the same reference number and a consecutive small letter, wherein for the elements of the first locking and release arrangement 118a the letter a, for the elements of the second locking and release arrangement 118b the letter b, and for the elements of the third locking and release arrangements 118c the letter c is used. Due to the identical structure of the locking and release arrangement 118a to 118c, hereinafter only the first locking and release arrangement 118a is described in every detail. The explanations with respect to the structure and function of the first locking and release unit 118a also apply to the further locking and release units 118b and 118c.

The locking and release arrangement 118a comprises clamping rolls 119a, 120a arranged between the leg 72a of the inner part 72 and the circular opening 80a of the outer part 80, a compression spring 121a arranged between the clamping rolls 119a, 120a, a first release element 122a which is arranged on the side of the clamping roll 119a opposite to the spring 121a, and a second release element 123a which is arranged on the side of the clamping roll 120a opposite to the spring 121a. The cross-sectional area between the leg 72a and the circular opening 80a tapers toward the leg end of the leg 72 so that the circumferential surfaces of the clamping rolls 119a, 120a rest against both the inside of the circular opening 80a and the outer leg 72a in the position shown in FIG. 5b so that when the inner part 72 is rotated in the first direction of rotation R1, the clamping roll 119a is clamped between the leg 72a and the circular opening 80a.

When the inner part 72 is rotated in the opposite second direction of rotation R2, the clamping roll 120a is clamped between the circular opening 80a and the leg 72a. Thus, a rotation of the inner part 72 with respect to the stationary outer part 80 is prevented by the clamping rolls 119a, 120a. The area between the leg 72a and the circular opening 80a in which the roll 119a is arranged, is referred to as first clamping area 124a, and the area between the leg 72a and the circular opening 80a in which the second clamping roll 120a is arranged is referred to as second clamping area 125a. The outer part 80, the inner part 72 and the first clamping roll 118a thus form a first locking unit and the outer part 80, the inner part 72 and the second clamping roll 125a form a second locking unit, wherein the first locking unit with the clamping roll 119a prevents a rotation of the inner part in the direction of rotation R1 and the locking unit with the clamping roll 120a prevents a rotation of the inner part 72 in the direction of rotation R2.

The clamping rolls 119a, 120a are pressed into their respective clamping area 124a, 125a by means of the spring 120a. When an output-side torque is applied to the driven shaft 55, which is in particular introduced via the foot receptacle 18 into the adjusting unit 30 and is transmitted by the telescopic tubes 51a, 51b to the driven shaft 55, the locking and release arrangements 118a to 118c block a rotary motion so that a rotation of the foot receptacle 18 is reliably prevented.

If, however, the rotation of the foot receptacle 18 is to be adjusted actively, then the second handwheel 36 is rotated so that via the rotary body 62 a rotation of the driving pins 64a to 64c about the axis of rotation Z takes place. Since the driving pins 64a to 64c, as already mentioned, are received in the respective bore 70a to 70c with play, a rotation of the driving pins 64a to 64c takes place in a first angular range about the axis of rotation Z without the inner part 72 being rotated as well. When the driving pins 64a to 64c are rotated in the direction of rotation R1, the first rotating spider 76 with the release pins 122a, 122b and 122c is rotated in the direction of rotation R1 so that the release pin 122a is moved from a neutral position into a release position and, in doing so, contacts the clamping roll 119a and pushes it out of its clamping area 124a. As a result, the clamping roll 119a can no longer block the rotation of the inner part 72. The movement of the inner part 72 in the direction of rotation R1 is thus released so that when the driving pins 64a to 64c are further rotated in the direction of rotation R1, these pins contact the walls of the bores 70a to 70c and rotate the inner part 72 together with the drive shaft 55. In contrast to the inner part 72, the rotating spider 76 is thus engaged with the driving pins 64a to 64c free from play in the direction of rotation R1 or engaged with substantially less play than the engagement of the driving pins 64a to 64c with the bores 70a to 70c.

When the inner part 72 is rotated, the driven shaft 55 formed integrally or in one piece with the inner part 72 is rotated so that the telescopic tubes 51a, 51b and the positioning head 42 arranged at the front end of the inner telescopic tube 51a are rotated together with the inner part 72. If no drive force is exerted any longer via the handwheel 36, the clamping roll 119a is pressed back into the clamping area 124a by the spring force of the spring 121a, as a result whereof the release element 122a is moved back into its neutral position so that a rotation of the inner part 72 in the direction of rotation R1 is reliably prevented subsequently by the clamping roll 119a even when a torque for rotation of the inner part 72 in the direction of rotation R1 is transmitted via the foot receptacle 10. When moving the release element 122a back, the rotating spider 76 is rotated in the second direction of rotation R2 so that all release elements 122a to 122c connected to the rotating spider 76 are moved back into their neutral positon.

When the second handwheel 36 is rotated in the direction of rotation R2, the rotary motion is transmitted substantially free from play via the rotary body 62 onto the driving pins 64a to 64c which are rotated in the direction of rotation R2 about the axis of rotation Z. As already mentioned, the driving pins 64a to 64c are received in the bores 70a to 70c of the inner part 72 with play so that the driving pins 64a to 64c can be rotated about an angular amount in the direction of rotation R2 until they hit the walls of the bores 70a to 70c and exert a driving torque on the inner part 72. During this angular displacement within the play, the driving pins 64a to 64c rotate about the second rotating spider 86 in the direction of rotation R2 so that the release element 123a contacts the clamping roll 120a and presses it out of the clamping area 125a against the spring force of the spring 121 a so that the inner part 72 can then be rotated in the direction of rotation R2 without the clamping roll 120a being clamped in the intermediate space between the leg 72a and the circular opening 80a of the outer part 80. As a result, a rotation of the inner part 72 and thus of the driven shaft 55 and the telescopic tube arrangement 55 connected to the driven shaft 55 in the direction of rotation R2 is possible by a mere actuation of the second handwheel 36. When the second handwheel 36 is no longer rotated in the direction of rotation R2 or if it is released, the spring 121a presses the clamping roll 120a back into the clamping area 125a, as a result whereof the clamping roll 120a presses the release element 123a from the release position back into its neutral position, wherein the entire rotating spider 86 is rotated in the direction of rotation R1 until all elements have the position shown in FIG. 5b. In this position, a rotation by means of a torque acting on the driven shaft 55 is again easily prevented by the locking and release units 118a to 118c. In the described rotation setting mode of the rotary drive 53, a rotation is thus only possible by means of a rotation of the second handwheel 36. The rotary drive 53 is, as will still be explained in detail in the following in connection with FIGS. 8 and 9, also operable in a rotation release mode which can be activated by means of the already mentioned actuating element 54. In this rotation release mode, both clamping rolls 119a, 120 are simultaneously moved out of their clamping areas 124a, 125a by means of the release elements 122a, 123a so that a rotary motion of the inner part 72 is released and the inner part 72 and all elements 55, 51a, 51b, 42, 44, 18 connected to the inner part 72 in a rotationally fixed manner can be rotated freely in both directions of rotation R1 and R2 both via the second handwheel 36 and via a torque acting on the driven shaft 55 in particular via the foot receptacle 18.

For this, the actuating element 54 is engaged with the rotation release disc 66 via the axially displaceable pins 56a to 56c (see pin 56a in FIGS. 5a to 5c) and press the rotation release disc 66 towards the inner part 72, as a result whereof the rotation release disc 66 simultaneously rotates the first rotating spider 76 in the direction of rotation R1 and the second rotating spider 86 in the direction of rotation R2, as a result whereof the release pins 122a to 122c, 123a to 123c connected to the rotating spiders 76, 86 are simultaneously moved from their neutral positions into their release positions and thus push the clamping rolls 119a to 119c and 120a to 120c simultaneously out of their clamping areas 124a to 124c, 125a to 125c. Via springs 74a, 74b and 74c arranged between the rotating spiders 76, 86 and the rotation release disc 66, of which springs only the spring 74a is visible in FIG. 5a, a restoring force for restoring the actuating element 54 into the position shown in FIG. 4 is developed so that when the actuating element 54 is no longer actuated, the rotation release disc 66 and the actuating element 54 connected via the pins 56a to 56c is moved back and a restoring of the release elements 122a to 122c, 123a to 123c by the spring force of the springs 121a to 121c takes place. As a result, the clamping rolls 119a to 119c, 120a to 120c are automatically moved back into their clamping areas 124a to 124c, 125a to 125c so that a rotation of the inner part 72 by an output-side torque is again easily and reliably prevented by the locking and release arrangements 118a to 118c.

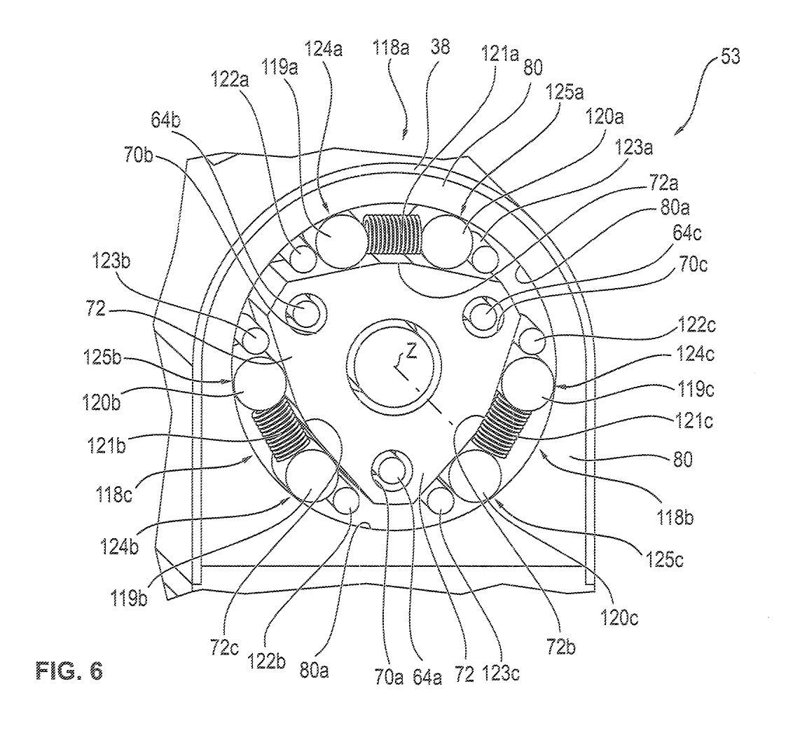

FIG. 6 shows a perspective illustration of a cross-section of the rotary drive 53 and through the housing 38 along the sectional line A-A according to FIG. 5a.

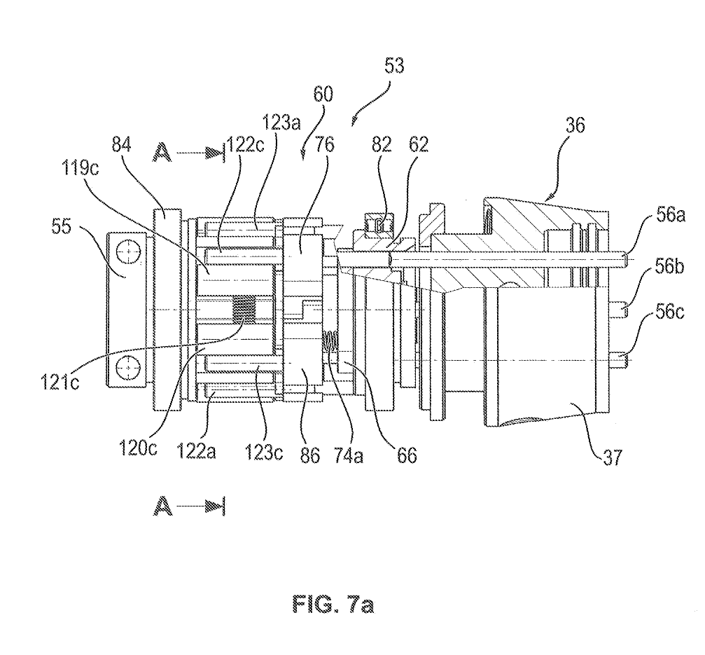

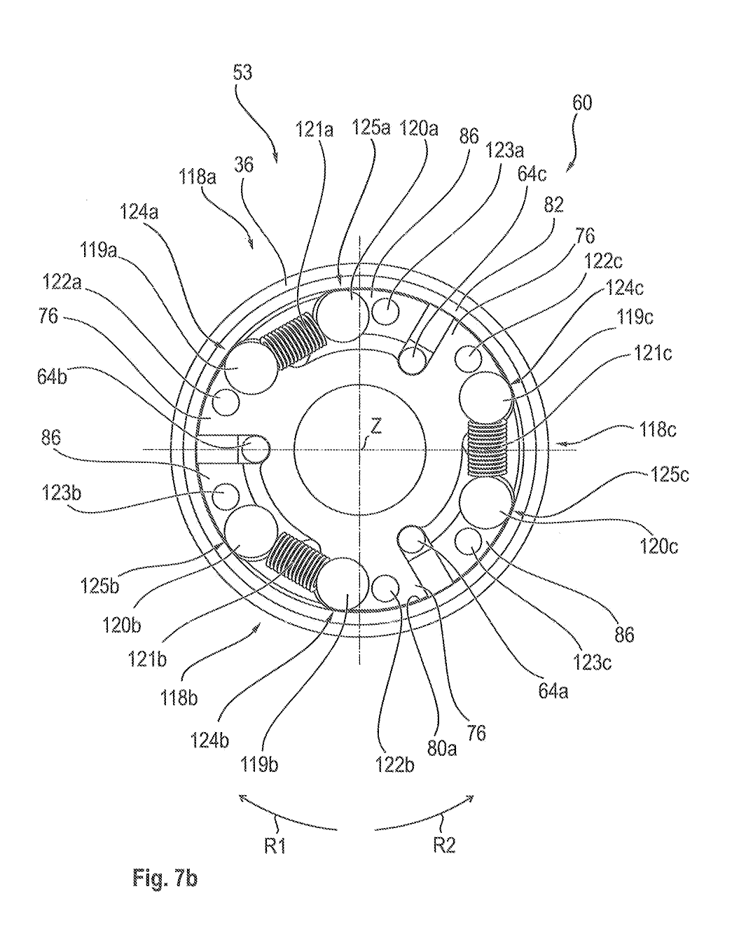

FIG. 7a is a further view of elements of the rotary drive 53 in a partially sectional illustration, similar to the one according to FIG. 5a, wherein the elements of the rotary drive 53 are illustrated in a different angular position compared to FIG. 5a. Further, in FIG. 7a in addition the hub 37 of the second handwheel 36 is illustrated without the three levers which can be screwed into the hub 37 for rotation thereof.

The three pins 56a to 56c project through the hub 37 and further extend through the rotary body 62. As a result, the pins 56a to 56c transmit a rotary motion of the handwheel 36 onto the rotary body 62. The pins 56a to 56c are arranged at equal angular distances about the axis of rotation Z. The driving pins 64a to 64c firmly connected to the rotary body 62 are arranged on a different circular path or offset to the pins 56a to 56c at equal angular distances about the axis of rotation Z. Thus, the rotary motion transmitted from the second handwheel 36 via the pins 56a to 56c onto the rotary body 62 is transmitted onto the driving pins 64a to 64c free from play, which then, as described, move the position of the release elements 123a to 123c, 124a to 124c dependent on the direction of rotation R1, R2 from a neutral position into a release position and subsequently rotate the inner part 72. As already mentioned, the pins 56a to 56c also serve to couple the actuating element 54 to the rotation release disc 66 in that the pins 56a to 56c are displaced by the actuating element 54 in axial direction toward the rotating spiders 76, 86 and displace the rotation release disc 66 toward the rotating spiders 76, 86.

In FIG. 7b, a sectional illustration of the arrangement with elements of the rotary drive 53 shown in FIG. 7a is illustrated along the sectional line A-A according to FIG. 7a. Both in FIG. 7a and in FIG. 7b, neither the outer part 80 nor the housing 38 are illustrated.

FIG. 8 shows a further perspective illustration of an arrangement with elements of the rotary drive 53, in which the elements for release of a rotary motion of the driven shaft 55 independent of the actuation of the second handwheel 36 are well visible. As already explained before, the pins 56a to 56c are moved toward the rotating spiders 76, 86 when the actuating element 54 is actuated and, in doing so, press the rotation release disc 66 in the direction of the rotating spiders 76, 86. For this, the rotation release disc 66 has rotation release elements 66a to 66c projecting in the direction of the rotating spiders 76, 86, which elements are pressed into areas 88a to 88c formed between arms 86a to 86c and 76a to 76c of the rotating spiders 76, 86 and thus rotate the rotating spiders 76, 86 against each other so that all release elements 122a to 122c, 123a to 123c are simultaneously moved from their neutral position into their release position. As a result, all clamping rolls 119a to 119c, 120a to 120c are moved out of their clamping areas 124a to 124c, 125a to 125c so that a free rotation of the driven shaft is also possible by means of output-side torques. When the actuating element 54 is moved back into its initial position, the pins 56a to 56c press the rotation release disc 66 no longer toward the rotating spiders 76, 86 so that the rotation release disc 66 is moved back toward the drive wheels 34, 36 by the springs 74a to 74c along the axis of rotation Z so that the rotation release elements 66a to 66c are no longer pressed between the arms 76a to 76c, 86a to 86c of the rotating spiders 76, 86, and the clamping rolls 119a to 119c, 120a to 120c are pressed back into the clamping areas 124a to 124c, 125a to 125c by the spring force of the springs 121a to 121c acting on them, and all release elements 122a to 122c, 123a to 123c are moved from their release position into their neutral position.

As an alternative to the described actuation of the actuating element 54 by pressing the actuating element toward the second handwheel or toward the front end of the adjusting unit 30, in another embodiment the actuation of the actuating element 54 can also be accomplished by pulling the actuating element toward the first handwheel 36 or toward the rear end of the adjusting unit 30. In particular, the rotation release disc 66 is turned over so that the rotation release elements 66a to 66c projects toward the actuating element 54. Further, the rotation release disc 66 is then arranged between the inner part 72 and the rotating spiders 76, 86.

The embodiments of the invention described above are provided by way of example only. The skilled person will be aware of many modifications, changes and substitutions that could be made without departing from the scope of the present invention. The claims of the present invention are intended to cover all such modifications, changes and substitutions as fall within the spirit and scope of the invention.

What is claimed is:

1. A device for the self-locking bidirectional drive of a medical treatment device, comprising
an inner part rotatable about an axis of rotation (Z),
a rotationally-fixed outer part having a circular opening which surrounds the inner part and is arranged concentrically about the axis of rotation (Z),
a driven shaft rotatable about the axis of rotation (Z), which driven shaft is connected to the inner part and is connectable to the treatment device,
a drive element for rotating the inner part together with the driven shaft about the axis of rotation (Z),
with a first clamping part and with a second clamping part which are arranged in an intermediate space between the inner part and the outer part, wherein
the outer part, the inner part and the first clamping part form a first locking unit for blocking the rotation of the inner part in a first direction of rotation (R1) and for releasing the rotation of the inner part in a second direction of rotation (R2),
that the outer part, the inner part and the second clamping part form a second locking unit for blocking the rotation of the inner part in the second direction of rotation (R2) and for releasing the rotation of the inner part in the first direction of rotation (R1),
that a first release element movable into a first release position by the drive element is provided, which release element prevents the blocking of the rotation of the inner part in the first direction of rotation (R1) by the first locking unit, and
that a second release element movable into a second release position by the drive element is provided, which release element prevents the blocking of the rotation of the inner part in the second direction of rotation (R2) by the second locking unit.

2. The device according to claim 1, wherein the drive element, when rotated in the first direction of rotation (R1), at first moves the first release element from a first neutral position into the first release position in which the first release element prevents the blocking of the rotation of the inner part in the first direction of rotation (R1) by the first locking unit and, when rotated further, rotates the inner part in the first direction of rotation (R1), and
that the drive element, when rotated in the second direction of rotation (R2), at first moves the second release element from a second neutral position into the second release position in which the second release element prevents the blocking of the rotation of the inner part in the second direction of rotation (R2) by the first locking unit and, when rotated further, rotates the inner part in the second direction of rotation (R2).

3. The device according claim 1, wherein the first clamping part is clampable in a first clamping area formed between the outer part and the inner part when the inner part is rotated in the first direction of rotation (R1), and
that the second clamping part is clampable in a second clamping area formed between the outer part and the inner part.

4. The device according to claim 1, wherein an elastically deformable element is arranged between the first clamping part and the second clamping part, which element presses the first clamping part into the first clamping area and the second clamping part into the second clamping area.

5. The device according to claim 3, wherein the elastic element is a spring, preferably a coil spring arranged between the clamping parts.

6. The device according to claim 4, wherein the first release element is arranged on the side of the first clamping part facing away from the elastically deformable element and the second release element is arranged on the side of the second clamping part facing away from the elastically deformable element.

7. The device according to claim 1, wherein when an output-side torque is applied to the driven shaft and/or to the inner part the first locking unit and the second locking unit prevent a rotation of the inner part and of the driven shaft without actuating the drive element.

8. The device according to claim 1, wherein the device enables a rotation of the driven shaft only by actuating the drive element without a separate rotation release.

9. The device according to claim 2, wherein the drive element is engaged with the inner part via at least one engagement element, wherein the engagement element is received in a recess of the inner part with play and/or wherein the engagement element is received in a recess of the drive element with play, wherein the play preferably has a value in the range between 0.5 mm and 5 mm.

10. The device according to claim 9, wherein the engagement element is a pin and the recess is a bore, wherein the pin projects into the bore and the diameter of the bore is preferably greater by a value in the range between 0.5 mm to 5 mm than the diameter of the pin.

11. The device according to claim 1, wherein the first release element and the second release element are operatively connected to the actuating element, and
that the actuating element, when actuated, moves the first release element into the first release position and the second release element into the second release position.

12. The device according to claim 1, wherein a first locking and release arrangement comprises at least the first locking unit, the second locking unit, the first release element and the second release element,
that the device comprises at least a second locking and release arrangement, wherein the structure and the function of the second locking and release arrangement correspond to the structure and the function of the first locking and release arrangement,
that the locking and release arrangements are arranged at equal angular distances about the axis of rotation (Z).

13. The device according to claim 12, wherein the device comprises three locking and release arrangements, the structure and function of which correspond to each other and which are arranged at equal angular distances about the axis of rotation (Z).

14. The device according to claim 12, wherein the first locking and release arrangement comprises at least the first release element, the second release element, the first clamping part and the second clamping part,
that the second locking and release arrangement comprises at least a third release element, a fourth release element, a third clamping part and a fourth clamping part,
that the third locking and release arrangement comprises at least a fifth release element, a sixth release element, a fifth clamping part and a sixth clamping part,
that the first, third and fifth release element are connected to each other such that they are jointly rotatable about the axis of rotation (Z), and
that the second, fourth and sixth release element are connected to each other such that they are jointly rotatable about the axis of rotation (Z),
wherein the first, third and fifth release element are jointly movable relative to the second, fourth and sixth release element, preferably by an angle in the range between 0.5° and 5°.

15. The device according to claim 14, wherein the actuating element, when actuated, simultaneously moves the first, third and fifth release element jointly in the first direction of rotation (R1) and the second, fourth and sixth release element in the second direction of rotation (R2),
so that the first release element contacts the first clamping part and prevents a clamping of the first clamping part in the first clamping area,
so that the second release element contacts the second clamping part and prevents a clamping of the second clamping part in the second clamping area,
so that the third release element contacts a third clamping part of the second locking and release arrangement and prevents a clamping of the third clamping part in a third clamping area formed between the inner part and the outer part,
so that the fourth release element contacts a fourth clamping part of the second locking and release arrangement and prevents a clamping of the fourth clamping part in a fourth clamping area formed between the inner part and the outer part,
so that the fifth release element contacts a fifth clamping part of the third locking and release arrangement and prevents a clamping of the fifth clamping part in a fifth clamping area formed between the inner part and the outer part, and/or
so that the sixth release element contacts a sixth clamping part of the third locking and release arrangement and prevents a clamping of the sixth clamping part in a sixth clamping area formed between the inner part and the outer part.

16. The device according to claim 2, wherein the first clamping part is clampable in a first clamping area formed between the outer part and the inner part when the inner part is rotated in the first direction of rotation (R1), and
that the second clamping part is clampable in a second clamping area formed between the outer part and the inner part.

17. The device according an to claim 2, wherein elastically deformable element is arranged between the first clamping part and the second clamping part, which element presses the first clamping part into the first clamping area and the second clamping part into the second clamping area.

18. The device according an to claim 3, wherein elastically deformable element is arranged between the first clamping part and the second clamping part, which element presses the first clamping part into the first clamping area and the second clamping part into the second clamping area.

19. The device according to claim 4, wherein the elastic element is a spring, preferably a coil spring arranged between the clamping parts.

20. The device according to claim 5, wherein the first release element is arranged on the side of the first clamping part facing away from the elastically deformable element and the second release element is arranged on the side of the second clamping part facing away from the elastically deformable element.
